(19) United States
(12) Patent Application Publication
(10) Pub. No.: US 2004/0022792 A1
(43) Pub. Date: Feb. 5, 2004

(54) Method of stabilizing proteins at low pH

(76) Inventors: Ralph Klinke, Sammanish, WA (US); Joshua R. Peterson, Seattle, WA (US)

Correspondence Address: IMMUNEX CORPORATION, LAW DEPARTMENT, 51 UNIVERSITY STREET, SEATTLE, WA, 98101

(21) Appl. No.: 10/464,206

(22) Filed: Jun. 17, 2003

Related U.S. Application Data
(60) Provisional application No. 60/389,375, filed on Jun. 17, 2002.

(51) Int. Cl. A61K 39/395; A61K 38/00

(52) U.S. Cl. 424/178.1; 514/12

(57) ABSTRACT

The present invention provides methods of stabilizing a protein at low pH by mixing the protein in a solution with one or more stabilizers in sufficient quantity to reduce the degree of aggregation of the protein at a low pH. In one embodiment, the stabilizers are selected from one or more of the following amino acids: glycine, leucine, lysine, alanine, methionine, aspartic acid and its salts, glutamic acid and its salts, arginine, tyrosine, and histidine. The methods of stabilizing a protein preparation find particular utility in stabilizing a protein during a low pH viral inactivation procedure.

### CROSS-REFERENCE TO RELATED APPLICATION

This application hereby claims the benefit under 35 U.S.C. §§ 119 (e) of U.S. provisional application serial No. 60/389,375, filed Jun. 17, 2002, the entire disclosure of which is relied upon and incorporated by reference.

### FIELD OF THE INVENTION

The present invention relates to the field of protein chemistry and more specifically to methods of stabilizing protein preparations during processing.

### BACKGROUND OF THE INVENTION

The purification of proteins for the production of biological or pharmaceutical products from various source materials involves a number of procedures. Therapeutic proteins may be obtained from plasma or tissue extracts, for example, or may be produced by cell cultures using eukaryotic or procaryotic cells containing at least one recombinant plasmid encoding the desired protein. The engineered proteins are then either secreted into the surrounding media or into the perinuclear space, or made intracellularly and extracted from the cells. A number of well-known technologies are utilized for purifying desired proteins from their source material. Purification processes include procedures in which the protein of interest is separated from the source materials on the basis of solubility, ionic charge, molecular size, adsorption properties, and specific binding to other molecules. Many of these procedures involve subjecting the protein of interest to low pH. For example, during affinity chromatography purification processes, certain proteins may be eluted from the column using a low pH buffer. Subjecting proteins to a low pH, however, may result in the denaturation and/or aggregation of the proteins.

In addition, one of the required steps during the preparation of pharmaceutical products is the inactivation of any viral or bacterial pathogens which may have originated from the source material. The goal of this step is to efficiently inactivate pathogens while retaining a high yield of biologically active proteins. Typically viral pathogens are inactivated using one of a set of standard treatments. These include heat treatment, carried out at a temperature and for a period of time sufficient to inactivate potential viruses such as heating the source material from between about 10 to 20 hours at 550 to 700 C. Other inactivation procedures include the use of solvent and detergents, exposure to radiation such as UV or IR radiation, nanofiltration techniques, treatment with virucidal agents including ethanol, lyophilization followed by heat treatment, mixing with a photosensitizing agent and irradiating, precipitation with polyethyleneglycol, or temporarily lowering the pH of a protein-containing solution. The challenge in all of these procedures is to minimize the destruction of the protein product while effectively inactivating viruses and other pathogens.

The present invention addresses and solves the problem of efficiently stabilizing proteins in order to prevent aggregation when subjecting a protein to low pH.

### SUMMARY OF THE INVENTION

The invention provides methods for stabilizing a protein held at a low pH. The method of the present invention involves the addition of a quantity of a stabilizer to a protein preparation sufficient to reduce protein aggregation when the preparation is held at a low pH. In one embodiment the method comprises adding a sufficient quantity of one or more amino acids to reach a final concentration of between about 1 mM to about 3 M, preferably between 1 mM and I M, and then subjecting the solution to a low pH, preferably a pH of about pH 4.0 or less, more preferably, between about pH 2.8 and about pH 4.0. The amino acids are selected from one or more of the following amino acids: glycine, alanine, leucine, lysine, methionine, phenylalanine, aspartic acid and the salts of aspartic acid, glutamic acid and the salts of glutamic acid, methionine, tyrosine, and histidine. Preferably, the amino acids are selected from one or more of the following amino acids: glycine, alanine, leucine, lysine, and methionine.

Additionally, stabilizers for the methods of present invention can be selected from a sugar or sugar derivative including sucrose, mannitol, and glycerol, or inorganic salt stabilizers such as sodium EDTA, NaCl, or CaCl₂. In one embodiment, one or more sugar or salt stabilizer can be combined with one or more amino acid stabilizer to reduce protein aggregation at low pH.

According to the method of the present invention, the concentration of the protein to be stabilized can vary from about 1 mg/ml to about 100 mg/ml in solution, preferably between about 5 mg/ml to about 30 mg/ml. The method of the present invention is useful for stabilizing any type of protein at low pH, but is particularly applicable to stabilizing recombinantly produced biologics during their purification process.

The invention also provides a stabilized protein composition at a pH of about 4.0 or less, which contains a protein preparation and a quantity of one or more stabilizers in solution sufficient to reduce protein aggregation compared with the composition containing no stabilizer. The stabilizers can be selected from one or more amino acids, present at a concentration of about 1 mM to 3 M, preferably about 1 mM to 1 M. The amino acids may be one or a combination of the following amino acids: glycine, alanine, leucine, lysine, methionine, phenylalanine, aspartic acid and the salts of aspartic acid, glutamic acid and the salts of glutamic acid, methionine, tyrosine, and histidine. In some aspects, the amino acids are one or more of the following: glycine, alanine, leucine, lysine, and methionine. Additionally, stabilizers can be selected from a sugar or sugar derivative including sucrose, mannitol, and glycerol, an inorganic salt such as sodium EDTA, NaCl, or CaCl₂, alone or combined with one or more amino acids. The protein concentration of the stabilized composition can vary from about 1 mg/ml to about 100 mg/ml according to the present invention, preferably between about 5 mg/ml to 30 mg/ml.

In another aspect, the present invention provides a method of stabilizing a protein preparation at a low pH during a virus inactivation step. The virus inactivation step involves reducing the pH of the stabilized protein preparation to a pH of about pH 4.0 or less for a period of time of at least 5 minutes or longer, preferably between about 30 minutes to about 60 minutes or longer.

The methods and compositions of the present invention are useful in any context in which a protein preparation is held at a low pH, including, but not limited to, the process of isolating and purifying the protein, the storage of a protein sample, and stabilizing the protein at low pH during a virus inactivation step.

### BRIEF DESCRIPTION OF THE FIGURES

FIG. 1 shows the degree of aggregation (% HMW) of an antibody that recognizes the epidermal growth factor receptor (EGFR) after exposure to pH 3.8 with no amino acids added, or with the addition of glycine, cysteine, glutamic acid, alanine, and lysine at the concentrations indicated on the X-axis. The % HMW (percent high molecular weight) material indicated on the Y-axis was determined by the percentage of aggregates plus dimers (areas under the respective peaks) compared with monomers (area under the peak) from a size exclusion chromatograph.

FIG. 2 shows a comparison of the degree of aggregation (% HMW) for an antibody that recognizes EGFR after exposure to pH 3.7 for 30 minutes with no amino acids added, or with the addition of each of the amino acids indicated on the X-axis.

FIG. 3 shows a comparison of the degree of aggregation (% HMW) for TNFR:Fc, a fusion protein having the soluble extracellular domain of TNF receptor fused to an Fc domain, after exposure to pH 3.5 with no amino acids added, or with the addition of each of the amino acids indicated on the X-axis.

FIG. 4 shows a comparison of the degree of aggregation (% HMW) for the fusion protein TNFR:Fc after exposure to pH 3.3 for 21.5 hours with no amino acids added, or with the addition of each of the amino acids indicated on the X-axis.

FIG. 5 shows a comparison of the degree of aggregation (% HMW) for a protein designated CD40 ligand (CD40L), a trimeric CD40 ligand fusion protein, after exposure to pH 3.7 with no amino acids added, or with the addition of each of the amino acids indicated on the X-axis.

### DETAILED DESCRIPTION OF THE INVENTION

The present invention is a method of reducing the amount of aggregation of a protein in a protein preparation when the preparation is subjected to a low pH. The method involves adding one or more stabilizers to a protein preparation while the pH of the protein preparation is reduced to a pH at which protein aggregation will otherwise occur. As used herein, the expression “stabilizing” is used interchangeably with “reducing protein aggregation”.

When a protein is in solution, lowering the pH of the solution may result in the disruption of the tertiary structure of the protein. The term “tertiary structure” of a protein refers to its three dimensional arrangement, that is, the folding of its secondary structural elements (the local conformation of the polypeptide backbone to form the α-helix, β-sheet, and turns), together with the spacial disposition of its sidechains. The disruption of the tertiary structure and partial unfolding of the protein can lead to the aggregation of the individual protein molecules.

As used herein the term “aggregation” refers to the formation of multimers of individual protein molecules through non-covalent or covalent interactions. Aggregation can be reversible or irreversible. When the loss of tertiary structure or partial unfolding occurs, hydrophobic amino acid residues which are typically hidden within the folded protein structure are exposed to the solution. This promotes hydrophobic-hydrophobic interactions between individual protein molecules, resulting in aggegation. Srisialam et al J Am Chem Soc 124 (9):1884-8 (2002), for example, has determined that certain conformational changes of a protein accompany aggregation, and that certain regions of specific proteins can be identified as particularly responsible for the formation of aggregates. Protein aggregation can be induced by heat (Sun et al. J Agric Food Chem 50(6): 1636-42 (2002)), organic solvents (Srisailam et al., supra), and reagents such as SDS and lysophospholipids (Hagihara et al., Biochem 41(3): 1020-6 (2002)). In vivo protein aggregation is a significant cause of disease, and is thought to occur as a result of improper folding or misfolding (Merlini et al., Clin Chem Lab Med 39 (11):1065-75 (2001)). Protein conformational diseases include Alzheimer's disease, Parkinson's disease, the prion encephalopathies, and Huntington's

disease. Aggregation is a significant problem in in vitro protein purification and formulation. Formation of aggregates can require solubilization with strong denaturating solutions followed by renaturation and proper refolding before biological activity is restored.

The presence and degree of aggregation of a particular protein molecule in a sample can be determined by suitable methods known in the art, such as size exclusion chromatography (SEC) as described in Example 1, for example, also known as gel filtration chromatography or molecular sieving chromatography. Another suitable method for determining the presence of aggregates in a sample is gel electrophoresis under non-denaturing conditions. The “gel” refers to a matrix of water and a polymer such as agarose or polymerized acrylamide. These methods separate molecules on the basis of the size of the molecule compared to the size of the pores of the gel. Other methods of measuring aggregation include hydrophobic interaction chromatography (HIC) and high performance liquid chromatography (HPLC). HIC separates native proteins on the basis of their surface hydrophobicity between the hydrophobic moieties of the protein and insoluble, immobilized hydrophobic groups on the matrix. Generally, the protein preparation in a high salt buffer is loaded on the HIC column. The salt in the buffer interacts with water molecules to reduce the solvation of the proteins in solution, thereby exposing hydrophobic regions in the protein which are then adsorbed by the hydrophobic groups on the matrix. The more hydrophobic the molecule, the less salt is needed to promote binding. Usually, a decreasing salt gradient is used to elute proteins from a column. As the ionic strength decreases, the exposure of the hydrophilic regions of the protein increases and proteins elute from the column in order of increasing hydrophobicity. See, for example, Protein Purification, 2d Ed., Springer-Verlag, New York, 176-179 (1988). HPLC (high performance liquid chromatography) provides a separation based on any one of adsorption, ion exchange, size exclusion, HIC or reverse phase chromatography. The separations are greatly improved, however through the use of high-resolution columns and decreased column retention times. See, for example, Chicz et al., Methods in Enzymology 182, pp. 392-421 (1990).

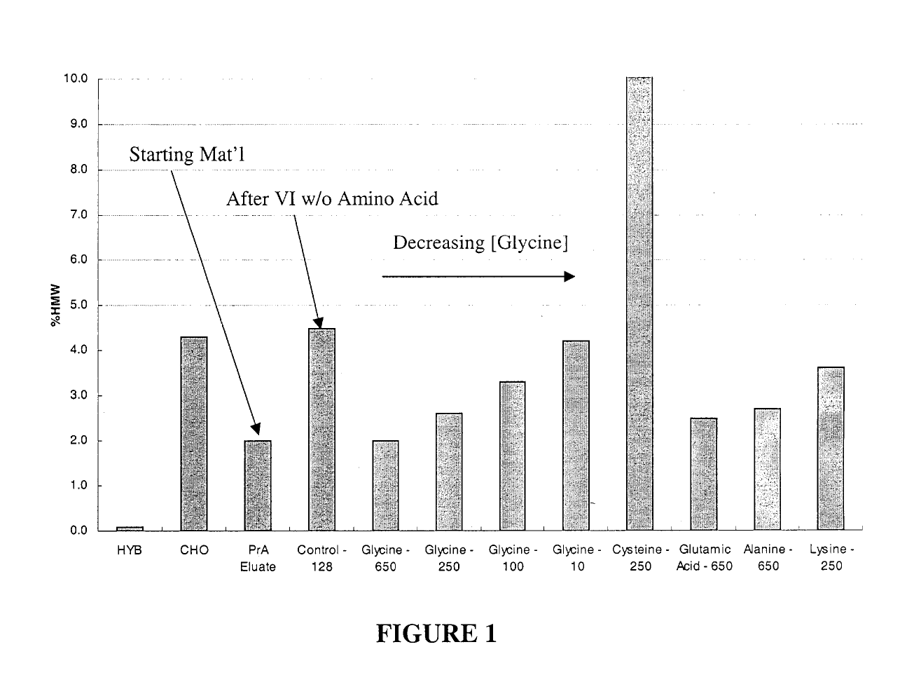

In one embodiment, the stabilizers employed according to the present invention are one or more free amino acids present in solution to a final concentration of between about 1 mM and about 3 M, preferably between about 1 mM and about 1 M. The preferred concentration of each amino acid stabilizer will depend in part on the solubility and particular properties of the amino acid chosen. For example, as shown in the table in Example 4 below, the final concentrations of stabilizing amino acids were varied from between 1 mM to 650 mM, depending on the solubility of the respective amino acid in water, and still showed effectiveness in reducing protein aggregation at low pH. The amino acid stabilizers are selected from one or a combination of the following: glycine, alanine, leucine, lysine, methionine, phenylalanine, aspartic acid and the salts of aspartic acid, glutamic acid and the salts of glutamic acid, methionine, tyrosine, and histidine. Preferably, the amino acid is one or a combination of the following: glycine, lysine, alanine, leucine and methionine. The effectiveness of an amino acid stabilizer at reducing protein aggregation will generally increase with increasing concentration of the amino acid. For example, as seen in FIG. 1, the effectiveness of glycine in reducing the degree of aggregation for an antibody increased dramatically with increasing concentration of glycine. While 10 mM glycine reduced the degree of aggregation of the antibody only marginally compared with the control sample (not exposed to low pH treatments), 100 mM glycine reduced aggregation by about 25 percent compared with the control, 250 mM glycine reduced aggregation by about 40 percent compared with the control, and 650 mM glycine reduced aggregation to the same level of aggregation as the control which was not exposed to low pH treatments.

As used herein, the term “amino acid” refers to the 20 standard α-amino acids as well as naturally occuring and synthetic derivatives. Amino acids are classed according to their sidechain as follows: nonpolar sidechain (glycine, alanine, valine, leucine, isoleucine, methionine, proline, phenylalanine, tryptophan), uncharged polar sidechains (serine, threonine, asparagine, glutamine, tyrosine, cysteine), charged polar sidechains (lysine, arginine, histidine, aspartic acid, glutamic acid). Amino acids are available as purified solids from a number of commercial sources. The amino acids can be mixed with the protein solution as a solid, in which the desired amount is dissolved into the protein solution, or as a concentrated aqueous solution in water or buffer which can be appropriately diluted into the protein preparation to the desired concentration.

Additionally, stabilizers can be selected from a sugar or sugar derivative, such as sucrose, mannitol, or glycerol. As used herein, the term “sugar” refers to monosaccharides such as glucose and mannose, or polysaccharides including disaccharides such as sucrose and lactose, as well as sugar derivatives including sugar alcohols and sugar acids. Sugar alcohols include mannitol, xylitol, erythritol, threitol, sorbitol and glycerol. An example of a sugar acid is L-gluconate. The sugar is provided at a concentration between about 10 mM and 3 M in solution, more preferably between about 100 mM and 1 M in solution. The stabilizer can also be selected from an inorganic salt such as sodium chloride, sodium ethylene diamine tetraacetic acid (EDTA), or calcium chloride, for example, at a concentration of between about 10 mM and 3 M, more preferably between about 100 mM and 1 M: In one embodiment of the present invention, a sugar or salt stabilizer may be combined with an amino acid stabilizer to improve the degree of stabilization for a particular protein. For example, 650 mM glycine can be combined with 400 mM NaCl to achieve a stabilized protein solution at low pH.

The protein preparation to be stabilized according to the present invention can vary from about 1 mg/ml to about 100 mg/ml, preferably between about 5 mg/ml to about 30 mg/ml, in an aqueous solution. As used herein the term “protein” is used interchangeably with the term “polypeptide” and is considered to be any chain of at least ten amino acids or more linked by peptide bonds. As used herein, the term “protein preparation” refers to protein in any stage of purification in an aqueous solution. The concentration of a protein preparation at any stage of purification can be determined by any suitable method. Such methods are well known in the art and include: 1) calorimetric methods such as the Lowry assay, the Bradford assay, and the colloidal gold assay; 2) methods utilizing the UV absorption properties of proteins; and 3) visual estimation based on stained protein bands in gels relying on comparison with protein standards of known quantity on the same gel. See, for example, Stoschek, Methods in Enzymol. 182:50-68 (1990).

For the purposes of the present invention a protein is “substantially similar” to another protein if they are at least 80%, preferably at least about 90%, more preferably at least about 95% identical to each other in amino acid sequence, and maintain or alter the biological activity of the unaltered protein. Amino acid substitutions which are conservative substitutions unlikely to affect biological activity are considered identical for the purposes of this invention and include the following: Ala for Ser, Val for Ile, Asp for Glu, Thr for Ser, Ala for Gly, Ala for Thr, Ser for Asn, Ala for Val, Ser for Gly, Tyr for Phe, Ala for Pro, Lys for Arg, Asp for Asn, Leu for Ile, Leu for Val, Ala for Glu, Asp for Gly, and the reverse. (See, for example, Neurath et al., The Proteins, Academic Press, New York (1979)).

A protein preparation can be stabilized at all stages of purification by the addition of the stabilizers according to the methods of the invention. Protein purification of recombinantly produced proteins typically includes filtration and/or differential centrifugation to remove cell debris and subcellular fragments, followed by separation using a combination of different chromatography techniques. These techniques separate protein mixtures on the basis of size, degree of hydrophobicity, charge, or affinity between the protein and a captured adsorbant. Some purification techniques expose protein samples to a low pH which is destabilizing. For example, a low pH elution buffer may be used for eluting certain proteins from an affinity purification column (see, for example, Ostrove, Methods in Enzymology 182:357-379 (1990)). According to the present invention, a stabilizer is preferably added to the protein preparation early in the purification process in order to reduce the amount of aggregation of the protein throughout the process, while not interfering with the purification procedure. Preferred stabilizers are the free amino acids. The amino acid stabilizers may be added to a protein preparation at any stage of purification, and can be removed from the preparation when desired through dialysis or filtration. The addition of the amino acid stabilizers most advantageously reduce protein aggregation when a protein purification step involves a reduction of the pH, particularly to a pH of about pH 4.0 or less. This is particularly applicable to such techniques, for example, as cation exchange chromatography (see, for example, Chicz et al., Methods in Enzymology 182: 392-421(1990), hydrophobic charge induction chromatography (HCIC) (see, for example, Schwart et al., J. Chromatog. 908 (1-2): 25-63 (2001), and Guerrier et al, Bioseparation 9(4): 211-221 (2000)), and mimetic affinity chromatography (see, for example, Murray et al., Anal. Biochem. 296 (1): 9-17(2001)).

In another aspect the invention provides a stabilized protein composition containing a protein and a quantity of at least one stabilizer sufficient to reduce aggregation of the protein at a low pH, preferably at a pH of about 4.0 or less. As used herein, the term “stabilized protein composition” or “stabilized protein preparation” refers to a protein preparation in an aqueous solution with the addition of the stabilizers described herein. The amino acid stabilizer is present at a concentration of between 1 mM and 3 M in solution, preferably between 1 MM and 1 M in solution. The amino acids stabilizers are selected from one or a combination of the following: glycine, alanine, leucine, lysine, methionine, phenylalanine, aspartic acid and the salts of aspartic acid, glutamic acid and the salts of glutamic acid, methionine, tyrosine, and histidine. Preferably, the amino acids are selected from one or a combination of glycine, lysine, aspartic acid, methionine, leucine, and alanine. The preferred concentration of amino acid used to stabilize the protein is determined from the solubility of the amino acid in water, as well as other considerations. For example, as can be seen from Example 3, glycine is capable of stabilizing a protein at low pH at concentrations as low as 10 mM, but the stabilization improves with increasing concentrations up to 650 mM, which approaches the limits of solubility for glycine. Additional stabilizers are selected from one or more sugars or sugar derivatives, or an inorganic salt, which stabilize alone or in combination with one or more amino acid stabilizers, at concentrations of between about 10 mM and 3 M, more preferably between about 100 mM to about 1 M. The concentration of the protein in the stabilized protein composition can vary from about 1 mg/ml to about 100 mg/ml, preferably from about 5 mg/ml to about 30 mg/ml. The protein is typically present in a buffered solution which has been adjusted to a low pH, preferably to a pH of about pH 4.0 more preferably to a pH between about pH 2.8 and about 4.0. As used herein, the term “buffer” or “buffered solution” refers to solutions which resist changes in pH by the action of its conjugate acid-base range. Examples of buffers that control pH at about pH 4.0 or less include acetate, succinate, citrate, or other mineral acid or organic acid buffers, and combinations of these.

In another aspect, the invention provides a method of stabilizing a protein preparation at a low pH during a virus inactivation procedure. Inactivation of viral pathogens which may have originated from the source material is a necessary step in the production of pharmaceutical products. Inactivation of viral and other pathogens originating from source materials may be accomplished a number of ways. These include heating to approximately 60° C. or higher for a number of hours (also referred to as “pasteurization”), subjecting the protein preparation to a solvent/detergent, subjecting the preparation to increased hydrostatic pressure followed by subjecting the sample to low temperatures (-15 to −20° C.), nanofiltration, or subjecting the protein preparation to low pH. Lowering the pH of the protein preparation is a quick and efficient way of inactivating viruses during the purification process. However, many proteins become aggregated at a low pH. The virus inactivation step requires that the protein preparation be titrated to a pH of about pH 4.0 or less, and held at this low pH for at least approximately 5 minutes or more, preferably between about 30 minutes to about 22 hours, more preferably between about 30 minutes to about 6 hours, more preferably between about 30 minutes to about 4 hours, more preferably between about 30 minutes to about 2 hours, most preferably between about 30 minutes to about 1 hour.

The method of stabilizing the protein preparation at a low pH during the viral inactivation step involves adding a sufficient quantity of one or more stabilizers as described herein to a protein preparation and then performing the viral inactivation step. The viral inactivation step can be performed at any stage of the purification process, however, the inactivation step is preferably performed early in the purification process. The protein can be present in a buffer, for example, acetate, succinate, citrate, other mineral or organic acid buffers or combinations of these, which are effective buffers at a pH of about 4 or less. The stabilized protein preparation can be titrated to a low pH with any number of acids including hydrochloric acid, succinic acid, and citric acid. According to the present invention, the stabilized protein preparation is titrated to a pH of about pH 4.0 or less, preferably between about pH 2.8 and about pH 4.0, and held at this pH for at least 5 minutes, preferably between about 30 to about 60 minutes or longer, to achieve viral inactivation. The stabilized protein preparation, however, will remain stable for a number of hours, depending on the protein, according to the methods of the present invention. After the viral inactivation step, the protein preparation may then be neutralized by increasing the pH using a base such as NaOH, or mixing with a buffered solution of a higher pH. Depending on the protein, the preparation can be neutralized to between about pH 4 and about pH 10, more commonly between about pH 5 and about pH 9. After neutralizing the pH of the preparation, the protein can then be further purified, formulated as a pharmaceutical, or lyophilized as is desired. Further purification steps can include SEC, HIC, ion-exchange chromatography, filtration, or any combination of these steps. In addition, proteins intended for pharmaceutical use can be subjected to testing throughout the production and purification processes, including testing for microbial contamination and/or extensive testing for the presence of various viruses.

Viral inactivation using exposure to low pH has in the past been considered particularly suitable for compositions containing human and animal immunoglobins (see, for example, Dr. Peter Neumann, “Workshop on Standards for Inactivation and Clearance of Infectious Agents in the Manufacture of Plasma Derivatives from Non-Human Source Materials for Human Injectable Use”, Oct. 25, 1999 (available from Office of Communication, Training and Manufacturers' Assistance, Center for Biologics Evaluation and Research, FDA, 1401 Rockville Pike, Rockville, Md. 20852-1448)). However, the present invention has demonstrated that low pH viral inactivation can be applied to a number of other types of proteins, since the stabilizers of the inventive method prevent or reduce additional aggregation of a protein when it is exposed to low pH.

The exact conditions required to achieve viral inactivation will vary in terms of time, temperature, and type of composition for a particular protein in order to reproducibly result in inactivation of particular viruses of concern. A pH of less than about pH 4.0, however, is considered necessary to achieve viral inactivation (Neumann, supra.). A number of commercial companies such as BioReliance Corporation, Rockville, Md. are available to assist in determining whether viral inactivation has been achieved.

According to the present invention, the method of stabilizing proteins at a low pH by adding an appropriate quantity of stabilizer to a solution is directed to stabilizing all types of proteins. The present invention is particularly directed to stabilizing protein-based drugs, also known as biologics, during all phases of their purification process as well as during the viral inactivation step at low pH. Typically biologics are produced recombinantly, using procaryotic or eukaryotic expression systems such as mammalian cells or yeasts. Recombinant production refers to the production of the desired protein by transformed host cell cultures containing a vector capable of expressing the desired protein. Methods and vectors for creating cells or cell lines capable of expressing recombinant proteins are described for example, in Ausabel et al, eds. Current Protocols in Molecular Biology, (Wiley & Sons, New York, 1988, and quarterly updates). Biologics are tested throughout their production and purification procedures for the presence of viruses and other microbial contaminants.

The method of stabilizing proteins at a low pH according to the present invention is particularly applicable to antibodies. As used herein, the term “antibody” refers to intact antibodies including polyclonal antibodies (see, for example Antibodies: A Laboratory Manual, Harlow and Lane (eds), Cold Spring Harbor Press, (1988)), and monoclonal antibodies (see, for example, U.S. Pat. Nos. RE 32,011, 4,902,614, 4,543,439, and 4,411,993, and Monoclonal Antibodies: A New Dimension in Biological Analysis, Plenum Press, Kennett, McKearn and Bechtol (eds.) (1980)). As used herein, the term “antibody” also refers to a fragment of an antibody such as F(ab), F(ab′), F(ab′)₂, Fv, Fc, and single chain antibodies which are produced by recombinant DNA techniques or by enzymatic or chemical cleavage of intact antibodies. The term “antibody” also refers to bispecific or bifunctional antibodies, which are an artificial hybrid antibody having two different heavy/light chain pairs and two different binding sites. Bispecific antibodies can be produced by a variety of methods including fusion of hybridomas or linking of Fab′ fragments. (See Songsivilai et al, Clin. Exp. Immunol. 79:315-321 (1990), Kostelny et al., J. Immunol. 148:1547-1553 (1992)). As used herein the term “antibody” also refers to chimeric antibodies, that is, antibodies having a human constant antibody immunoglobin domain is coupled to one or more non-human variable antibody immunoglobin domain, or fragments thereof (see, for example, U.S. Pat. No. 5,595,898 and U.S. Pat. No. 5,693,493). Antibodies also refers to “humanized” antibodies (see, for example, U.S. Pat. No. 4,816,567 and WO 94/10332), minibodies (WO 94/09817), and antibodies produced by transgenic animals, in which a transgenic animal containing a proportion of the human antibody producing genes but deficient in the production of endogenous antibodies are capable of producing human antibodies (see, for example, Mendez et al., Nature Genetics 15:146-156 (1997), and U.S. Pat. No. 6,300,129). The term “antibodies” also includes multimeric antibodies, or a higher order complex of proteins such as heterdimeric antibodies. “Antibodies” also includes anti-idiotypic antibodies including anti-idiotypic antibodies against an antibody targeted to the tumor antigen gp72; an antibody against the ganglioside GD3; or an antibody against the ganglioside GD2.

One exemplary antibody stabilized according to the present invention is an antibody that recognizes the epidermal growth factor receptor (EGFR), referred to as “an antibody against EGFR” or an “anti-EGFR antibody”, described in U.S. Pat. No. 6,235,883, which is herein incorporated by reference. An antibody against EGFR includes but is not limited to all variations of the antibody as described in U.S. Pat. No. 6,235,883. Many other antibodies against EGFR are well known in the art, and additional antibodies can be generated through known and yet to be discovered means. One exemplary antibody against EGFR is a fully human monoclonal antibody capable of inhibiting the binding of EGF to the EGF receptor. The stabilization of an anti-EGFR antibody at low pH according to the present invention is described herein in Examples 2, 3, and 4.

The invention is also particularly applicable to proteins, in particular fusion proteins, containing one or more constant antibody immunoglobin domains, preferably an Fc domain of an antibody. The “Fc domain” refers to the portion of the antibody that is responsible for binding to antibody receptors on cells. An Fc domain can contain one, two or all of the following: the constant heavy 1 domain (C_{H}1), the constant heavy 2 domain (C_{H}2), the constant heavy 3 domain (C_{H}3), and the hinge region. The Fc domain of the human IgG1, for example, contains the C_{H}2 domain, and the C_{H}3 domain and hinge region, but not the CHI domain. See, for example, C. A. Hasemann and J. Donald Capra, Immunoglobins: Structure and Function, in William E. Paul, ed. Fundamental Immunology, Second Edition, 209, 210-218 (1989). As used herein the term “fusion protein” refers to a fusion of all or part of at least two proteins made using recombinant DNA technology or by other means known in the art.

An example of an Fc-containing protein capable of being stabilized according to the present invention is tumor necrosis factor receptor-Fc fusion protein (TNFR:Fc). As used herein the term “TNFR” (tumor necrosis factor receptor) refers to a protein having an amino acid sequence that is identical or substantially similar to the sequence of a native mammalian tumor necrosis factor receptor, or a fragment thereof, such as the extracellular domain. Biological activity for the purpose of determining substantial similarity is the capacity to bind tumor necrosis factor (TNF), to transduce a biological signal initiated by TNF binding to a cell, and/or to cross-react with anti-TNFR antibodies raised against TNFR. A TNFR may be any mammalian TNRF, including murine and human, and are described in U.S. Pat. No. 5,395,760, U.S. Pat. No. 5,945,397, and U.S. Pat. No. 6,201,105, all of which are herein incorporated by reference. TNFR:Fc is a fusion protein having all or a part of an extracellular domain of any of the TNFR polypeptides including the human p55 and p75 TNFR fused to an Fe region of an antibody, as described in U.S. Pat. No. 5,605,690, which is incorporated herein by reference. An exemplary TNFR:Fc is a dimeric fusion protein made of the extracellular ligand-binding portion of the human 75 kDa tumor necrosis factor receptor linked to the Fe portion of the human IgG I from natural (non-recombinant sources), as described in U.S. Pat. No. 5,705,364 and U.S. Pat. No. 5,721,121, both of which are incorporated herein by reference. The stabilization of this exemplary TNFR:Fc at low pH according to the present invention is described in Example 4.

Additional proteins capable of being stabilized by the methods of the present invention include differentiation antigens (referred to as CD proteins) or their ligands or proteins substantially similar to either of these. Such antigens are disclosed in Leukocyte Typing VI (Proceedings of the VIth International Workshop and Conference, Kishimoto, Kikutani et al., eds., Kobe, Japan, 1996). Similar CD proteins are disclosed in subsequent workshops. Examples of such antigens include CD27, CD30, CD39, CD40, and ligands thereto (CD27 ligand, CD30 ligand, etc.). Several of the CD antigens are members of the TNF receptor family, which also includes 41BB ligand and OX40.

An exemplary ligand capable of being stabilized according to the present invention is a CD40 ligand (CD40L). The native mammalian CD40 ligand is a cytokine and type II membrane polypeptide, having soluble forms containing the extracellular region of CD40L or a fragment of it. As used herein, the term “CD40L” refers to a protein having an amino acid sequence that is identical or substantially similar to the sequence of a native mammalian CD40 ligand or a fragment thereof, such as the extracellular region. As used herein, the term “CD40 ligand” refers to any mammalian CD40 ligand including murine and human forms, as described in U.S. Pat. No. 6,087,329, which is herein incorporated by reference. Biological activity for the purpose of determining substantial similarity is the ability to bind a CD40 receptor. A exemplary embodiment of a human soluble CD40L is a trimeric CD40L fusion protein having a 33 amino acid oligomerizing zipper (or “leucine zipper”) in addition to an extracellular region of human CD40L as described in U.S. Pat. No. 6,087,329. The 33 amino acid sequence trimerizes spontaneously in solution. The stabilization of CD40L at low pH according to the present invention is described in Example 4 below.

In addition, a number of other proteins are capable of being stabilized at low pH according to the present invention, particularly any protein of commercial, economic, pharmacologic, diagnostic, or therapeutic value. Such proteins may be monomeric or multimeric. These proteins include, but are not limited to, a protein or portion of a protein identical to, or substantially similar to, one of the following proteins: a flt3 ligand, erythropoeitin, thrombopoeitin, calcitonin, Fas ligand, ligand for receptor activator of NF-kappa B (RANKL), TNF-related apoptosis-inducing ligand (TRAL), thymic stroma-derived lymphopoietin, granulocyte colony stimulating factor, granulocyte-macrophage colony stimulating factor, mast cell growth factor, stem cell growth factor, epidermal growth factor, RANTES, growth hormone, insulin, insulinotropin, insulin-like growth factors, parathyroid hormone, interferons, nerve growth factors, glucagon, interleukins 1 through 18, colony stimulating factors, lymphotoxin-β, tumor necrosis factor, leukemia inhibitory factor, oncostatin-M, and various ligands for cell surface molecules ELK and Hek (such as the ligands for eph-related kinases or LERKS). Descriptions of proteins that can be stabilized according to the inventive methods may be found in, for example, Human Cytokines: Handbook for Basic and Clinical Research, Vol. II (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook (A. W. Thompson, ed., Academic Press, San Diego, Calif., 1991).

Additional proteins capable of being stabilized according to the present invention are receptors for any of the above-mentioned proteins or proteins substantially similar to such receptors or a fragment thereof such as the extracellular domains of such receptors. These receptors include, in addition to both forms of tumor necrosis factor receptor (referred to as p⁵⁵ and p75) already described: interleukin-1 receptors (type 1 and 2), interleukin-4 receptor, interleukin-15 receptor, interleukin-17 receptor, interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL, and receptors that comprise death domains, such as Fas or apoptosis-inducing receptor (AIR). Proteins of interest also includes antibodies which bind to any of these receptors.

Proteins of interest capable of being stabilized according to the present invention also include enzymatically active proteins or their ligands. Examples include polypeptides which are identical or substantially similar to the following proteins or portions of the following proteins or their ligands: metalloproteinase-disintegrin family members, various kinases, glucocerebrosidase, superoxide dismutase, tissue plasminogen activator, Factor VIII, Factor IX, apolipoprotein E, apolipoprotein A-I, globins, an IL-2 antagonist, alpha-i antitrypsin, TNF-alpha Converting Enzyme, ligands for any of the above-mentioned enzymes, and numerous other enzymes and their ligands. Proteins of interest also include antibodies that bind to the above-mentioned enzymatically active proteins or their ligands.

Additional proteins of interest capable of being stabilized according to the present invention are conjugates having an antibody and a cytotoxic or luminescent substance. Such substances include: maytansine derivatives (such as DM1); enterotoxins (such as a Staphlyococcal enterotoxin); iodine isotopes (such as iodine-125); technium isotopes (such as Tc-99m); cyanine fluorochromes (such as Cy5.5.18); and ribosome-inactivating proteins (such as bouganin, gelonin, or saporin-S6). Examples of antibodies or antibody/cytotoxin or antibody/luminophore conjugates contemplated by the invention include those that recognize the following antigens: CD2, CD3, CD4, CD8, CD11a, CD 14, CD18, CD20, CD22, CD23, CD25, CD33, CD40, CD44, CD52, CD80 (B7.1), CD86 (B7.2), CD147, 1L-4, IL-5, IL-8, IL-10, IL-2 receptor, IL-6 receptor, PDGF-β, VEGF, TGF, TGF-β2, TGF-β1, VEGF receptor, C5 complement, IgE, tumor antigen CA125, tumor antigen MUCI, PEM antigen, LCG (which is a gene product that is expressed in association with lung cancer), HER-2, a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA4 (which is a cytotoxic T lymphocyte-associated antigen), Fc-γ-1 receptor, HLA-DR 10 beta, HLA-DR antigen, L-selectin, IFN-γ, Respiratory Syncitial Virus, human immunodeficiency virus (HIV), hepatitis B virus (HBV), Streptococcus mutans, and Staphlycoccus aureus.

The method of the present invention is particularly useful for stabilizing a protein during protein purification processes, including but not limited to stabilizing a protein during the viral inactivation step. The methods of stabilizing proteins are additionally useful for temporary storage, and analytical procedures which may involve lowering the pH of the protein in solution.

The invention having been described, the following examples are offered by way of illustration, and not limitation.

### Example 1

### Size Exclusion Chromatography to Determine Degree of Aggregation

Size exclusion chromatography (SEC) was used to quantify the degree of aggregation or percentage of large aggregates, dimers, or monomers of a given protein before and after the protein sample was subjected to a certain condition such as low pH.

Samples of an antibody against EGFR (epidermal growth factor receptor) (described in U.S. Pat. No. 6,235,883) were analyzed by size exclusion chromatography (SEC) using silcia gel columns (TSK G3000SWxl, Toyo Haas) to determine the degree of aggregation of the protein under non-denaturing conditions. Samples of protein were diluted to 2 mg/mL in running buffer (100 mM NaH₂PO₄, 150 mM NaCl, pH 6.8). 10 uL was injected onto the column. A flow rate of 1 mL/min was used and absorbance at 220 nm was monitored. The SEC chromatogram for this antibody, for example, indicated the presence of various levels of aggregation including aggregates (530 Kd), dimers (330 Kd), and monomers (165Kd). The area under the curves on the chromatogram can be integrated to determine the relative quantities of aggregate, dimer (both refered to as high molecular weight forms), and monomer in the solution.

### Example 2

### Purification Protocol for an Antibody Using Glycine

The amino acid glycine was used to reduced aggregation of an antibody against EGFR in solution at various times during purification, as demonstrated in the following protocol. The steps in the following protocol were carried out at temperatures of approximately 17° C. to 25° C.

Cells producing antibodies such as hybridomas or recombinant cell cultures were grown up, and the antibodies were secreted directly in the medium. The cultures were harvested and the cells removed by differential centrifugation or ultrafiltration. The supernatant was loaded directly an affinity chromatography column. The column was eluted in the presence of 650 mM glycine solution in 25 mM sodium citrate buffer at about pH 4.1. The elution pool was titrated with 1M citric acid to pH 3.7. The solution was held at pH 3.7 for at least 30 minutes or longer as a viral inactivation step. The pH was then neutralized to pH 6.0±0.2 with IN NaOH. The antibody was then further purified. The presence of glycine was found to reduce the degree of aggregation of the antibody throughout the purification process.

### Example 3

### Stabilizing Effect of Glycine and other Amino Acids on an Antibody

An antibody against EGFR was subjected to a low pH viral inactivation step during a purification process. The antibody was eluted from an affinity chromatography column at pH 4.1 in 25 mM sodium citrate buffer. The concentration of the antibody was determined to be 17 mg/ml. Various aliquots of this antibody preparation in 25 mM sodium citrate buffer were prepared with no amino acids added, and with increasing concentrations of glycine (10 mM, 100 mM, 250 mM, and 650 mM), glutamic acid (650 mM), alanine (650 mM), and lysine (250 mM) added. The various aliquots were titrated to pH 3.7+/−0.1 with 1 M citric acid and aliquots kept at this pH for 30 minutes. After 30 minutes the aliquots were neutralized to pH 6.0+/−0.2 with 1 N NaOH. The samples were then subjected to SEC, the percentage of high molecular material determined, and the results shown in FIG. 1. FIG. 1 shows the stabilization of the antibody when subjected to pH 3.8 with the amino acids tested at the concentrations shown, with the exception of cysteine. As can be seen from FIG. 1, the stabilization of the antibody improved with increasing glycine concentrations of up to 650 mM. In fact, the addition of 650 mM of glycine was able to prevent any additional aggregation upon low pH treatment when compared to the untreated material (first column, FIG. 1).

### Example 4

### Stabilizing Effect of Various Amino Acids on Protein Solutions Titrated to Low pH for Three Proteins

The following proteins were subjected to low pH with and without the presence of various amino acids: (1) an antibody against EGF receptor, described in Examples 2 and 3 above, (2) soluble form of tumor necrosis factor receptor extracellular domain fused to an Fc domain (TNFR:Fc), and (3) a trimeric CD40 ligand fusion protein. The amino acids were obtained in solid form (Sigma-Aldrich), and were added to the solution as a solid to obtain the final concentrations as shown below.

| | |
|---|---|
| β-alanine | 650 mM |
| L-leucine | 100 mM |
| DL-methionine | 150 mM |
| L-phenylalanine | 150 mM |
| Glycine | 650 mM |
| L-cysteine | 650 mM |
| L-(-) tyrosine | 1 mM |
| DL-aspartic acid | 15 mM |
| L-glutamic acid | 30 mM |
| L-lysine | 650 mM |
| L-arginine | 500 mM |
| L-histidine | 150 mM |

The procedures followed for each protein are given as follows.

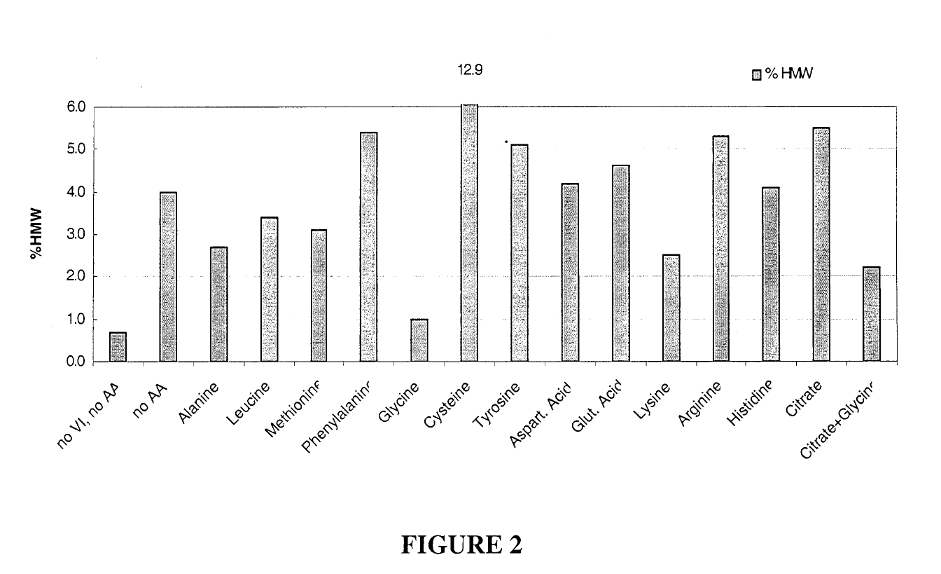

(1) For the antibody, a preparation of 15 mg/ml in 25 mM sodium citrate buffer was divided into a number of aliquots and the appropriate amount of amino acids added to achieve the indicated final concentration in the table above. In addition to the amino acids listed above, citrate was added to one aliquot a final concentration of 250 mM. The aliquots were titrated to pH 3.7 with 1 M citric acid, and held for 30 minutes at room temperature. All aliquots were then neutralized to pH 6.0 with 1 N NaOH. The degree of aggregation for each aliquot was then determined using SEC as described in Example 1 above. The results are shown in FIG. 2. FIG. 2 shows stabilization of the antibody at pH 3.7 by alanine, leucine, methionine, glycine, and lysine, but not phenylalanine, cysteine, tyrosine, aspartic acid, glutamic acid, arginine, and histidine. Glycine was particularly effective at stabilizing the antibody.

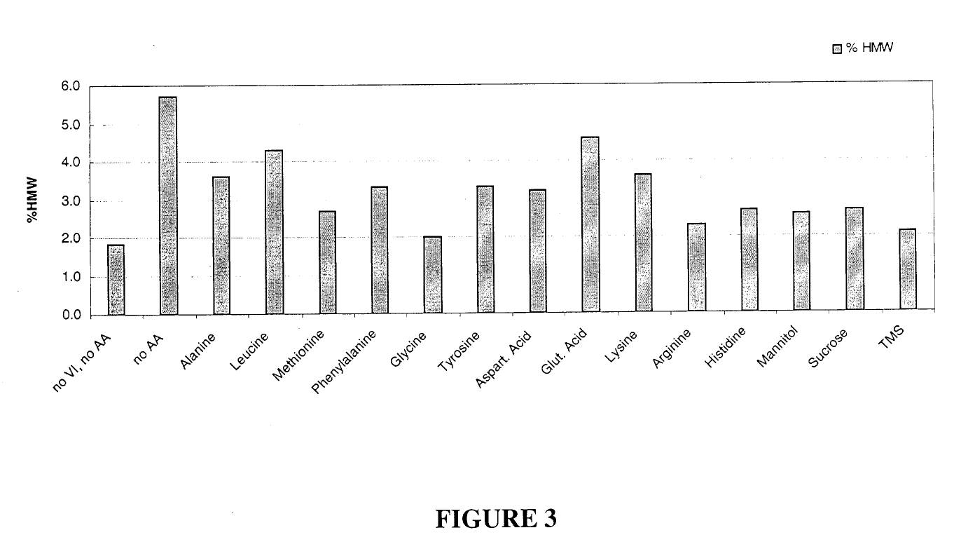

For TNFR:Fc, the protein preparation of 20 mg/ml in 50 mM sodium acetate buffer divided into a number of aliquots and the appropriate amount of amino acid was added to achieve the indicated final concentration. The aliquots were titrated with 1 N HCl to pH 3.5 and held at this pH for 30 minutes at room temperature. The solutions were then neutralized to pH 7.5 with I N NaOH. The degree of aggregation for each aliquot was then determined using SEC. The results are given in FIG. 3. FIG. 3 shows that alanine, leucine, methionine, phenylalanine, glycine, tyrosine, aspartic acid, glutamic acid, lysine, arginine, histidine, and the non-amino acids mannitol (4% w/v)), sucrose (1% w/v), and TMS (25 mM Tris-mannitol(4%)-sucrose (1%) buffer, titrated to pH 3.5) have some stabilizing effect. Glycine at 650 mM is particularly effective.

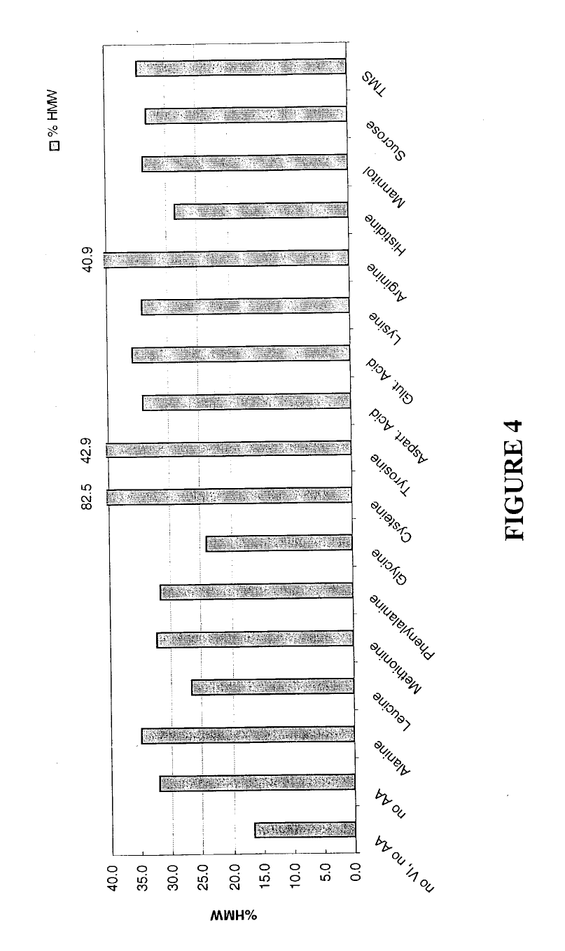

An additional experiment was done to assess the stabilization of TNFR:Fc over a longer period of time. The protein preparation at a concentration of 15 mg/ml in 50 mM sodium acetate buffer was divided into a number of aliquots and the appropriate amount of amino acid was added to each aliquot to achieve the indicated final concentration. The aliquots were titrated with 1 N HCl to pH 3.3 and held at this pH for 21.5 hours at approximately 25° C. The aliquots were then neutralized to pH 7.5 with 1 N NaOH. The degree of aggregation for each aliquot was then determined using SEC. The results are given in FIG. 4. FIG. 4 shows that the degree of aggregation can be reduced by adding leucine (by about 20% compared with the treated sample with no amino acids), histidine (by about 12.5%), and in particular glycine (by about 25%) even during exposure to low pH for a period as long as 21.5 hours.

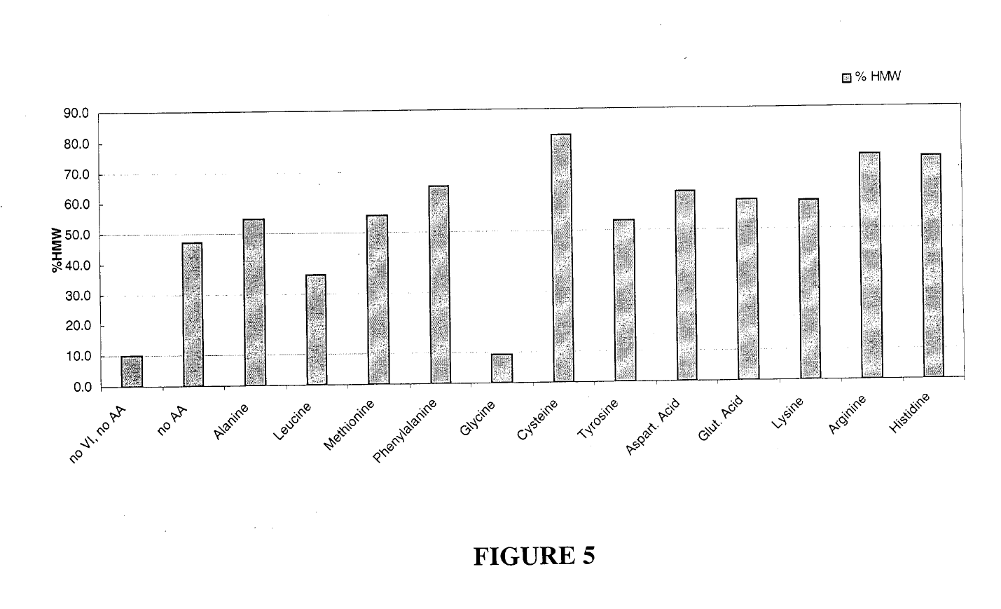

For CD40L, a protein preparation of 10 mg/ml in BDS solution was divided into a number of aliquots with an appropriate amount of amino acid stabilizer added to achieve the indicated concentration. The aliquots were titrated to pH 3.7 with 1 N HCl, and held at this pH for 30 minutes, and the solutions were then neutralized to pH 6.5 with 1 N NaOH. The degree of aggregation of the protein samples in the aliquots was determined using SEC. The results are shown in FIG. 5. It can be seen from FIG. 5 that alanine, leucine, methionine, glycine, tyrosine, aspartic acid, glutamic acid, and lysine have some degree of stabilizing effect on CD40L, with leucine and glycine having the most pronounced effect. Again, the addition of glycine was able to prevent any additional aggregation upon exposure to low pH when compared to the untreated material.

The present invention is not to be limited in scope by the specific embodiments described herein, which are intended as single illustrations of individual aspects of the invention, and as functionally equivalent methods and components that are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A method of stabilizing a protein in an aqueous preparation at a low pH comprising adding a quantity of one or more amino acids to the preparation sufficient to reduce the degree of aggregation of the protein to less than that of the protein without the amino acid, and reducing the pH of the preparation to between about pH 2.8 and about pH 4.0, wherein the amino acids are selected from the group consisting of glycine, leucine, lysine, alanine, methionine, aspartic acid and its salts, glutamic acid and its salts, arginine, tyrosine, and histidine.

2. The method of claim 1, wherein the final amino acid concentration is between about 1 mM and about 3 M.

3. The method of claim 1, wherein the final amino acid concentration is between about 1 mM and about 1 M.

4. The method of claim 1, wherein the amino acids are selected from the group consisting of glycine, leucine, lysine, alanine, and methionine.

5. The method of claim 1, wherein the concentration of the protein in the preparation is between about 1 mg/ml and about 100 mg/ml.

6. The method of any one of claims 1 through 5, wherein the protein is a recombinant Fc-containing fusion protein, a differentiation antigen or a ligand of the differentiation antigen.

7. The method of claim 1, wherein the pH of between about pH 2.8 and about 4.0 is maintained for 30 minutes or longer.

8. The method of claim 1, further comprising the step of raising the pH of the preparation to between about pH 4 and about pH 10.

9. The method of claim 8, wherein the pH is raised by the addition of sodium hydroxide solution.

10. The method of claim 8, wherein the pH is raised by the addition of a sodium citrate solution.

11. The method of claim 1, further comprising testing for microbial contamination.

12. The method of claim 1, further comprising purifying the protein.

13. The method of claim 1, further comprising formulating the protein.

14. The method of claim 1, further comprising lyophilizing the protein.

15. The method of claim 4, wherein the protein being stabilized is an antibody against EGF receptor.

16. The method of claim 1, wherein the protein being stabilized is a TNFR:Fc fusion protein.

17. The method of claim 1, wherein the protein being stabilized is a CD40L, and the amino acids are selected from the group consisting of alanine, leucine, methionine, glycine, tyrosine, aspartic acid, glutamic acid, and lysine.
